(19) United States
(12) Patent Application Publication
(10) Pub. No.: US 2004/0110667 A1
(43) Pub. Date: Jun. 10, 2004

(54) Carrier system for cyclosporin pharmaceutical compositions

(76) Inventor: Edwards E. Linn, Fairfield, NJ (US)

Correspondence Address: KLAUBER & JACKSON, 411 HACKENSACK AVENUE, HACKENSACK, NJ, 07601

(21) Appl. No.: 10/357,114

(22) Filed: Feb. 3, 2003

Related U.S. Application Data
(60) Provisional application No. 60/430,755, filed on Dec. 4, 2002.

(51) Int. Cl. A61K 38/13

(52) U.S. Cl. 514/11

(57) ABSTRACT

A homogeneous cyclosporin composition containing a pharmaceutically effective amount of cyclosporin in association with a pharmaceutical carrier, said carrier comprising a drug solubilizing effective amount of mono or diester of propylene glycol of lauric acid with monoester content of at least 90% by weight, a non-ionic surfactant and a dispersing agent. The composition described herein provides greater solubility of cyclosporin and cyclosporin capsule shell stability.

### DOMESTIC PRIORITY CLAIM

The priority-is claimed of U.S. Provisional Application No. 60/430,755, filed on Dec. 4, 2002, which is hereby incorporated by reference herein in its entirey.

### FIELD OF THE INVENTION

The present invention relates to a microemulsion concentrate carrier system for pharmaceutical compositions containing cyclosporin.

### BACKGROUND OF THE INVENTION

The Cyclosporins are a class of structurally distinctive, cyclic, poly-N-Methylated undecapeptides, possessing common pharmacological, particularly immunosuppressive, anti-inflammatory and/or anti-parasitic (e.g. anti-malarial and other anti-protozoic) activity. The first of the Cyclosporins isolated was the naturally occurring fungal metabolite Cyclosporin, also known as cyclosporin A or Cyclosporine.

Cyclosporin is highly lipophilic and only sparingly soluble in water, but dissolves readily in organic solvents, such as methanol, ethanol, chloroform and the like. Due to its limited solubility in water, the bioavailability of orally administered cyclosporin is extremely low. Poor bioavailability may lead to ineffective therapy and a need for the use of higher dosages with a resultsing in the potential for undesirable side effects. Providing an effective therapeutic concentration of the drug in the body when administered orally or by routes requiring transmembrane absorption has been a difficult problem. Research has focused on finding a cyclosporin preparation effective for oral administration that provides both uniform dosage and effective bioavailability of the active component.

Such oral preparations have heretofore included the combination of cyclosporin with a surfactant, an oil and a co-surfactant. For example, U.S. Pat. No. 4,388,307 discloses a liquid formulation of cyclosporin that includes at least one of: a transesterification product of a natural or hydrogenated vegetable oil and a polyalkylene polyol; a saturated fatty acid triglyceride; or a mono-or diglyceride. Ethanol is preferred as a solubilizing agent. However, since this liquid formulation is administered as an aqueous solution, it is both inconvenient and difficult to administer in a uniform dosage.

Belgian Pat. No. 895,724, which relates to the use of Cyclosporin in the treatment of multiple sclerosis, also describes two oral formulations suitable for the administration of this particular compound. Both of these are based on the commercial Cyclosporin (SANDIMMUN™) drinking solution modified to suit the particular cyclosporin active ingredient. The first comprises 5-10% Cyclosporin, 10-12% ethanol, 30-40% MAISINE™, about 4% CREMOPHORE™ and 15-30% LABRAFIL™. This corresponds to the composition of the liquid oral formulation of SANDIMMUN™, but with the replacement of the natural vegetable oil component with MAISINE™ which is transesterification products of corn oil with glycerol, and the introduction of a minor percentage of the tensides (e.g.,CREMOPHORE™). The ratio of Cyclosporin to tenside in the disclosed composition is 1: 0.4-0.8. Inasmuch as ethanol is a key component of the formulation, it does not make any suggestion to replace ethanol as co-solvent/co-surfactant.

However, the use of ethanol as well as other solvents such as 1,2,propylene glycol systems creates several problems. Since ethanol permeates the gelatin shell of the capsule and is volatile, even at room temperature, the constitutional ratio of the contents of the soft capsules may greatly vary during storage. The resulting reduced ethanol content may in turn result in crystallization of the cyclosporin, and this results in a significant variation in the bioavailability of cyclosporin when administered to an animal. The variation in cyclosporin concentrate in these types of formulations makes it quite difficult to determine the dosage needed to provide a desired therapeutic effect. Moreover, when solvents such as ethanol, 1,2-propylene glycol and liquid polyethylene glycols are utilized in gelatin capsules, these solvents have a tendency to absorb moisture, thereby rendering brittle the shell walls, especially those in hard gelatin capsules, and thereby resulting in leakage of the contents of the capsules during storage or shipment. Moreover, one of the biggest drawbacks using hydrophilic components, as in U.S. Pat. No. 5,342,625, has been the potential of re-precipitation of the drug from the formulation when it comes into contact with aqueous systems, such as in the stomach or intestine after ingestion by the mammal.

There is a need to prepare formulations of cyclosporin which minimize the number of components that are to be administered to the patient. There is also a need to prepare formulations of cyclosporin having greater solubility particularly greater drug solubility and capsule shell stability which is resistance to brittleness, and using components considered as GRAS, and offering a desirable pharmacokinetic property such as bioavailability, and having the ease of manufacturing.

The present inventor has found such a system which overcomes the problem of limited solubility and resultant poor bioavailability of cyclosporin. Unlike the formulation used heretofore, it has been found that the use of a carrier system comprising mono/diester of propylene glycol laurate, most importantly with mono ester content not less than 90% (e.g., Lauroglycol 90) and a non-ionic surfactant and a dispersing agent to overcome the problems described herein. The propylene glycol ester of lauric acid with mono ester content greater than 90%, is less hygroscopic compared to other propylene glycol esters such as propylene glycol ester of C8 fatty acids (Capmul PG8) as well as glycerol esters such as monoglycerides of C8 fatty acids (Capmul MCM) and therefore provides greatest physical stability of the capsule with respect to brittleness and drug stability on storage (see hygroscopicity data in as GRAS according to CFR21 Section 172.856. In addition, the propylene glycol ester of lauric acid containing mono ester greater than 90% exhibited highest solubility of cyclosporin (thereby minimizing the size and number of capsules that are administered to the patient each day) as compared to others formulated by using propylene glycol mono esters (between 50 to 70%) of lauric acid (Table 2)

It has been found that by employing the above defined carrier system, it is possible to obtain cyclosporin formulations which do not require any solvent or co-solvent such as ethanol, propylene glycol, and the like, in amounts effective to solubilize the drug. Therefore, problems associated with these solvents, as mentioned hereinabove, are eliminated by the present invention. Thus, the compositions of the present invention are more stable compared to one associated with alcohols, in which the alcohols are utilized and present in amounts effective for dissolving cyclosporin.

Due to the greater solubility of cyclosporin in the present formulation, it has a concomitant advantage: For example, the size of the capsule for the delivery of unit doses containing cyclosporin is reduced in the present invention, providing greater patient acceptance and compliance. Moreover, if the oral dosage form is a capsule, there is an excellent compatibility and physical stability of the propyleneglycol momo/dilaurate with mono ester greater than 90% by weight in the hard or soft shell gelatin capsules, thereby preventing brittleness and leakage of the formulation during storage. Furthermore, the present pharmaceutical composition is a pre-concentrate which forms an emulsion, preferably forms a fine emulsion upon contact with the aqueous fluids (e.g., water or aqueous fluids in GI. tract) which provides higher and uniform bioavailability of the drug. This characteristic further helps reduce the intra- and inter-subject variability associated with the absorption of the lipophilic active component, as well as minimize the effect of food on the absorption and bioavailability of cyclosporin in mammals.

### SUMMARY OF THE INVENTION

The present invention is directed to a pharmaceutically acceptable carrier to be used in association with cyclosporin, said pharmaceutically acceptable carrier including: a monoester or diester of propylene glycol laurate with mono ester content of at least 90% by weight; a non-ionic surfactant; and a dispersing agent, the composition forming a fine emulsion on exposure to fluids such as aqueous fluid or gastrointestinal secretions. The present invention is also directed to a method of enhancing the solubility of cyclosporin in a pharmaceutical composition containing the aforementioned components, comprising thoroughly mixing the cyclosporin with a cyclosporin solubilizing agent consisting essentially of propylene glycol mono/diester of lauric acid with mono ester not less than 90% by weight.

### BRIEF DESCRIPTION OF THE FIGURES

FIG. 1 shows hygroscopicity data for various liquid excipients.

### DETAILED DESCRIPTION OF THE INVENTION

As indicated hereinabove, an aspect of the present invention relates to a pharmaceutically acceptable carrier system in association with cyclosporin in a pharmaceutical formulation. It is preferred that the formulation be used in an oral dosage form, e.g., in hard or soft gelatin capsules (or capsules made of other materials such as starch, cellulose or its derivatives, etc.).

Cyclosporin, which is used as the pharmaceutically active ingredient in the composition according to the present invention, is a cyclic peptide compound having useful immunosuppressive activity and anti-inflammatory activity. Although various cyclosporins, such as cyclosporin A and the like can all be used as the cyclosporin component in the present invention, cyclosporin A is preferred. It is present in the composition of the present invention in pharmaceutically effective amounts. These amounts are well-known in the prior art. For example, when treating chronic inflammations or provoking an immunosuppressive effect, it is preferred that the daily dose ranges from about 3 mg/kg to about 50 mg/kg in a preferred embodiment the cyclosporin is present in amounts ranging from about 2.5 to about 20% by weight of the pharmaceutical composition.

As defined by the present invention, cyclosporin is associated with a carrier system comprising a mono/diester of propylene glycol laurate with mono ester not less than 90% by weight, a non ionic surfactant and dispersing agent. The composition consisting of propylene glycol mono laurate with monoester greater than 90% is important for greater compatibility with the cyclosporin in getting maximum solubility, additionally due to its GRAS status (CFR 172.856) it is more suitable for human oral use.

Moreover, it is most preferred that the fatty acids utilized in the present invention are regarded as Generally Recognized As Safe (GRAS) by the FDA for oral use.

The carrier system used in the present invention are commercially available or are prepared by art-recognized techniques. They are commercially available from Condea, N.J. USA. It is also commonly known as Lauroglycol 90.

In the composition of the present invention, it is preferred that the cyclosporin solubilizing agent is present in amounts sufficient to solubilize cyclosporin. As indicated hereinabove, the present carrier system solubilizes cyclosporin. In a preferred composition, cyclosporin is present in 2.5 to 20% by weight, the carrier system (A) described herein is present in the weight percentage of 45-80%, more preferably in 50-70% and most preferably 55-60% (all percentages by weight). The nonionic surfactant is present in weight percentage of 5-60%, more preferably 20-50% and most preferably 30-40% and dispersing agent 1-10% most preferably about 2-5% (by weight).

The present inventor has found that the carrier system, when used in the amounts indicated hereinabove, greatly enhances the solubility of cyclosporin. As a result, the pharmaceutical composition, when combined with an effective amount of nonionic surfactant, in accordance with the present invention, provides an isotropic homogeneous mixture which exhibits excellent bioavailability of the cyclosporin in vivo. In addition, the present formulation also exhibits greater encapsulation stability.

For example, the solubilization of cyclosporin in a composition containing Lauroglycol 90 was found to be superior as compared to any formulations containing hydrophobic and/or lipophilic materials in terms of physical and chemical stability.

More specifically, cyclosporin is extremely soluble in the composition of the present invention. The present formulation does not require the presence of ethanol, which is typically used for purposes of solubilizing cyclosporin and for the purpose of oral administration, as disclosed in the prior formulations.

Another essential component of the carrier system in the pharmaceutical composition of the present invention is the water soluble nonionic surfactant. It is preferred that the surfactant has a HLB (Hydrophilic Lipophilic Balance) greater than 10, more preferably greater than 12 and most preferably greater than 14. The surfactant is capable of forming a stable emulsion, for example, preferably a fine emulsion and more preferably a microemulsion, of the present composition when it is brought into contact with aqueous fluids, such as that in the G.I. tract. Examples of the preferred surfactants according to the present invention include polyoxyethylene products of hydrogenated vegetable oils, polyethoxylated castor oils or polyethoxylated hydrogenated castor oil, polyoxyethylene-sorbitan-fatty acid esters, polyoxyethylene castor oil derivatives and the like, for example, NIKKOL HCO-50™, NIKKOL HCO-35™, NIKKOL HCO-40™, NIKKOL HCO-60™ (from Nikko Chemicals Co. Ltd.); CREMOPHORE™(from BASF) such as CREMOPHORE RH40™, CREMOPHORE RH60™, CREMOPHORE EL™.

The surfactant can include more than one nonionic surfactant, as defined hereinabove, including any of the above-mentioned surfactants alone or in combination with one or more surfactants. In the composition according to the present invention, it is preferred that the fatty acid and surfactant be used in a weight ratio described herein.

The third important component is a dispersing agent, which forms instantly very fine emulsion on exposure to gastrointestinal fluid. The dispersing agent is glyceryl triacetate (known as triacetin) or glyceryl tricaprylate/caprate (known as Miglyol 812)

Additives and diluents normally utilized in the pharmaceutical arts can optionally be added to the pharmaceutical composition and especially the carrier. These include thickening, granulating, dispersing, flavoring, sweetening, coloring, and stabilizing agents, including pH stabilizers, other excipients, anti-oxidants (e.g., tocopherol, BHA, BHT, TBHQ, tocopherol acetate, ascorbyl palmitate, ascorbic acid propyl gallate, and the like), preservatives (e.g., parabens), and the like.

It is preferred that an anti-oxidant is present in the pharmaceutical composition of the present invention. It is preferred that the antioxidant is present in at least about 0.1% by weight of the pharmaceutical composition, and more preferably from about 0.1% to about 2% by weight of the pharmaceutical.

The present pharmaceutical composition is prepared by uniformly and thoroughly mixing the carrier, cyclosporin, and the surfactant together at room temperature or at slightly elevated temperature, such as temperatures up to about 40° C. until a clear solution is obtained, and then cooled to room temperature. The other additives indicated hereinabove are then thoroughly admixed therewith. The cyclosporin remains in solution and does not crystallize or precipitate out.

An essential aspect of the pharmaceutical formulation of the present invention is that it forms an emulsion, e.g., a fine emulsion, when placed in contact with water or an aqueous medium. The emulsion, e.g., fine emulsion formed is thermodynamically stable when it comes into contact with water or aqueous media such as the GI fluids of the mammals. Thus, the present formulation not only increases the solubility of the cyclosporin in the pharmaceutical carrier but also enhances the solubility thereof in the treated mammal and facilitates uniform absorption thereof in the treated mammal.

In addition, the fine emulsion (particle size as determined by laser light scattering to be <1 micron) formed by the carrier system of the present formulation upon contact with water gives rise to a faster onset of action.

Compositions of the present invention are preferably administered to mammals, such as dogs, cats, horses, pigs, mice, rats and especially humans. It is preferred that the pharmaceutical compositions of the present invention are administered orally in capsule, tablet, liquid-oral, powder, or the like or liquid for parenteral composition for intramuscular or intravenous administration. In a preferred embodiment, the invention provides a composition in a form appropriate or adapted for oral administration, in particular, in oral unit dosage form, e.g., in the form of tablets, capsules, drink solutions or dry powder for reconstituting; or a sohxlet form prepared by standard techniques known in the art, such as by spray coating on deposition. Especially suitable unit dosage forms for oral administration include encapsulated forms, e.g., soft or hard gelatin encapsulated forms, which is the preferred oral dosage form.

Oral unit dosage forms in accordance with the present invention will suitably comprise from 5 to 400 mg and more preferably from 20 to 200 mg, e.g., 25, 50, 100, 125, 150, or 200 mg of cyclosporin. The dosage of the drug and the number of times administered to the patient will vary depending on several factors, the age of the patient, the severity of the condition of the patient, past medical history, among other factors, and will be determined by the physician in his sound discretion.

When the composition of the present invention is prepared in the form of a soft or hard capsule, the composition may be encapsulated in a gelatin shell which contains any conventional plasticizer. As the plasticizer can be included in the gelatin capsule shell, one or more selected from the group consisting of glycerine, sorbitol, hexanetriol propylene carbonate, hexane glycol, sorbitans, tetrahydrofuryl alcohol ether, diethylene glycol mono ethyl ether, 1,3-trimethyl-2-imidazolidone, dimethylisosorbide, etc. can be used without any limitation. However, it should be understood that the plasticizer which can be used in the present invention is not restricted to those mentioned above.

Capsule preparation according to the present invention can be prepared in a conventional machine by encapsulating the resulting pre-concentrates of the emulsion of the present invention, e.g., the microemulsion pre-concentrate with or without the above-mentioned pharmaceutically acceptable additives.

Since ethanol is preferably not present, especially in amounts sufficient to solubilize the cyclosporin, there is less risk of precipitating or crystallizing the cyclosporin in the pharmaceutical composition. If ethanol were present, and if it were present in the amounts usually found in cyclosporin formulations described in the prior art, it would evaporate even when standing at room temperature, thereby causing possible crystallization and/or precipitation of the cyclosporin. The absence of ethanol in these amounts in the present formulation prevents possible crystallization and precipitation of the cyclosporin, thereby ensuring dosage uniformity, accurate blood levels of cyclosporin and consistent therapeutic performance.

Moreover, there is no need for special precaution and procedure for the manufacturing, packaging and handling requirement during the preparation, storage and shipping of the product since ethanol is not present.

In addition, the compositions of the invention exhibit improved stability on storage as compared with compositions based on the use of ethanol or equivalent alkanols and are, in particular, better adapted, e.g., for presentation in capsule, e.g. hard or soft gelatin capsule form. Compositions in accordance with the present invention which are free or substantially free of ethanol have the particular advantage of eliminating or substantially reducing packaging difficulties, e.g. in relation to the packaging of soft gelatin encapsulated forms.

The present pharmaceutical invention forms a more stable system and is capable of holding a larger amount of cyclosporin than prior art formulations.

Moreover, the present formulation can also be administered as a parenteral preparation for intra-muscular or even intravenous use.

Thus the present pharmaceutical formulation has several advantages. It exhibits (1) an enhanced solubility of cyclosporin, thereby providing for higher drug loading and reducing the size of oral unit dosage of same (e.g., the size of the capsule will be reduced); (2) greater and uniform bioavailability; (3) better storage stability; (4) reduced inter and intra-subject variability, and (5) minimal effect of food on the oral absorption of the drug. And most importantly (6) greater physical stability of the capsule towards brittleness. Moreover, the present formulation utilizes GRAS materials for oral use. Furthermore, administration of the present formulation in the reduced size dosage forms, such as capsules will facilitate greater patient acceptance and compliance. Administration of the present formulation in the reduced size dosage forms, such as capsules, will facilitate greater patient acceptance and compliance.

As used herein, the term a “drug” refers to “cyclosporin”, thus, the two terms are used interchangeably without changing the meaning thereof.

Moreover, the term “aqueous medium” as used herein, includes water, fluids containing water and in vivo media in mammals, such as the aqueous fluid present in the G.I. tract thereof.

Unless indicated to the contrary the % utilized are in weight percentages.

The following examples further illustrate the present invention.

### EXAMPLE 1

| Ingredient | mg |
|---|---|
| Cyclosporin | 100 |
| Lauroglycol 90 | 280 |
| Cremophore EL | 225 |
| Miglyol 812 | 15 |
| Alpha tocopherol | 5 |
| Total | 625 |

This example utilized the above ingredients in the amounts indicated. The cyclosporin was dissolved in propylene glycol mono/diester of lauric acid with mono ester not less than 90% by weight. Polyoxyethylene 35 castor oil (Cremophore EL) which was the surfactant was then added and mixed therewith for a few minutes at room temperature until the solution was homogenous. Then triacetin and alpha tocopherol are added and mixed until a homogeneous mixture is obtained.

The solution was then stored up to 6 weeks to ensure that no crystallization occurred.

To verify that an emulsion was formed, 1 part of the formulation was added to 10 parts of water and stirred gently. The drug did not precipitate or crystallize and the resulting solution formed a fine emulsion.

The formulation is ready for encapsulation into a capsule.

### EXAMPLE 2

The procedure of Example 1 is followed except the formulation contains the following:

| Ingredient | mg |
|---|---|
| Cyclosporin | 25 |
| Lauroglycol 90 | 80 |
| Cremophore EL | 50 |
| Miglyol 812 | 7.5 |
| Alpha tocopherol | 2.5 |
| TOTAL | 165 |

### EXAMPLE 3

The procedure of Example 1 is followed except the formulation contains the following:

| Ingredient | mg |
|---|---|
| Cyclosporin | 100 |
| Lauroglycol 90 | 300 |
| Cremophore EL | 230 |
| Miglyol 12 | 10 |
| Alphatocopherol | 5 |
| TOTAL | 645 |

### COMPARATIVE EXAMPLES

To show the improved solubility and greater capsule stability towards brittleness of the cyclosporin in the pharmaceutical composition of the present invention, the formulation of the present invention with propylene glycol mono/diester of lauric acid (Lauroglycol 90) with not less than 90% of mono ester was compared with that of the pharmaceutical formulation prepared with propylene glycol mono/diester of caprylic acid (Capmul PG8) and glyceryl mono caprylate (Capmul MCM8)

The formulations were prepared by mixing the components described hereinbelow at room temperature until a clear solution was formed. The resulting formulations were stored for 4 weeks and then compared. The results are tabulated hereinbelow:

TABLE 1

| | Inventive | Comparative | Comparative |
|---|---|---|---|
| Composition | Example | Example I | Example II |
| CyclosporinA | 100 | 100 | 100 |
| Lauroglycol90 | 300 | | |
| Capmul PG8 | | 300 | |
| Capmul MCM8 | | | 300 |
| Cremophore EL | 230 | 230 | 230 |
| Miglyol812 | 10 | 10 | 10 |
| Alphatocopherol | 5 | 5 | 5 |
| Results of | | | |
| stability | | | |
| Intial | No | No | No |
| | brittleness | brittleness | brittleness |
| 1M 25° C. + | No | Slight | More |
| 60% RH | brittleness | brittleness | brittleness |
| 3M 25° C. + | No | More | Severe |
| 60% RH | brittleness | brittleness | brittleness |

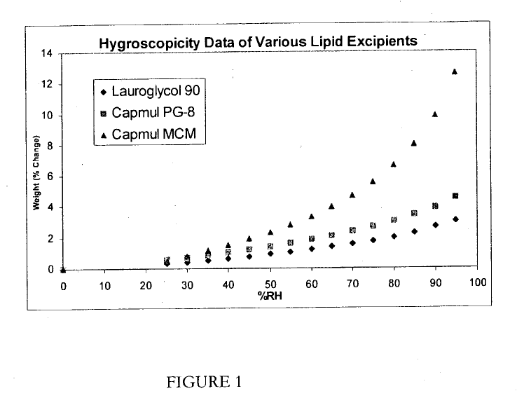

As clearly shown by the data, in the present inventive formulation, the cyclosporin capsule maintained physical stability in inventive formulation, however capsules containing comparative formulation (Capmul PG8 or Capmul MCM) became brittle. This further emphasizes the importance of using a right kind of ester of propylene glycol such as with lauric acid containing at least 90% by weight monoester. Thus, the present formulation is more suitable for use in pharmaceutical formulations for it enhances the ability of the cyclosporin to remain solubilized for extended shelf life. The hypothesis was further supported by hygroscopicity data as shown in FIG. 1.

Importance of mono ester of lauric acid in cyclosporin formulation: It is important to note that at least 90% of the monoester of lauric acid is required as disclosed in this invention.

To prove the importance of this percentage of mono ester present in propylene glycol mono ester of lauric acid for solubilizing and forming fine emulsions of cyclosporin, the following experiments were carried out

TABLE 2

| | Composi- | Composi- | Composi- |
|---|---|---|---|
| Composition | tion 1 | tion 2 | tion 3 |
| Cyclosporin A | 100 | 100 | 100 |
| Lauroglycol 90 | 300 | 135 | — |
| Lauroglycol FCC | — | 165 | 300 |
| Cremophore EL | 230 | 230 | 230 |
| Miglyol 812 | 10 | 10 | 10 |
| Alpha tocopherol | 5 | 5 | 5 |
| % of mono ester in | >90% | = 65% | = 50% |
| propyleneglycol ester | | | |
| of lauric acid | | | |
| Observation | Clear | Hazy | Crude |
| | solution | suspension | suspension |

The results above clearly showed that 90% of mono ester was the most suitable one in solubilizing and forming a clear solution of cyclosporin. On the other hand, the formulation containing 65% propylene glycol monoester of lauric acid (Example 2 above) and 50% of propylene glycol ester of lauric acid (Example 3 above) are not effective. This further proves the uniqueness of 90% propylene glycol mono ester of lauric acid in solubilizing the drug and offering a more stable formulation in terms of capsule shell stability.

The above preferred embodiments and examples are given to illustrate the scope and spirit of the present invention. These embodiments and examples will make apparent to those skilled in the art other embodiments and examples. These other embodiments and examples are within the contemplation of the present invention.

Therefore, the present invention should be limited only by the appended claims.

We claim:

1. A pharmaceutical composition comprising a pharmaceutically effective amount of cyclosporin in a carrier vehicle, said carrier vehicle including at least a mono/diester of propylene glycol lauric acid in which the monoester content is at least 90% by weight, a non-ionic surfactant having HLB value of greater than 10, and a dispersing agent.

2. The pharmaceutical composition of claim 1 where cyclosporin is present in 1-20%, the carrier vehicle present in 45-80%, the non-ionic surfactant in 5-60%, and the dispersing agent in 1-10%, all percentages being by weight.

3. The pharmaceutical composition of claim 1 wherein cyclosporin is present at about 2.5 to 10%, the carrier vehicle at about 55-60%, the non-ionic surfactant at about 20-40%, and the dispersing agent at about 2-5% by weight.

4. The pharmaceutical composition of the claim 1 wherein the carrier vehicle carrier comprises a mixture of mono and diesters of propylene glycol of lauric acid and the mono ester content is not less than 90% by weight.

5. The pharmaceutical composition of claim 1 wherein the non-ionic surfactant has HLB greater than 10 and is selected from the group consisting of polyoxyethylene products of hydrogenated vegetable oils, polyethoxylated castor oils, polyethoxylated hydrogenated castor oil, polyoxyethylene-sorbitan-fatty acid esters, polyoxyethylene castor oil derivatives and blends of two or more of the foregoing.

6. The pharmaceutical composition of claim 1 wherein the dispersing agent is glyceryl tricaprylate or glyceryl triacetate.

7. The pharmaceutical composition of claim 1 further comprising may contain one or more antioxidants, the antioxidants selected from the group consisting of BHA, BHT, and Alpha tocopherol.

8. The pharmaceutical composition of claim 7 wherein the antioxidant is present in the amount of about 0.01% to about 2% by weight.

9. The pharmaceutical composition of claim 1 which can be formulated as an solution.

10. The composition of claim 1 which can be formulated as hard or soft capsule.

11. A method for the treatment or prevention of protozoal infection by administering to a subject an effective inflammation treating or preventing dose of a pharmaceutical composition according to claim 1.

12. A method for the treatment of inflammation by administering to a subject an effective inflammation treating or preventing dose of a pharmaceutical composition according to claim 1.
